(19) United States
(12) Patent Application Publication
(10) Pub. No.: US 2006/0167405 A1
(43) Pub. Date: Jul. 27, 2006

(54) BODILY FLUID SPACE ENTRY DETECTION

(75) Inventors: Alan D KING, Burlington, CT (US); James M MUSKATELLO, Southington, CT (US)

Correspondence Address: WOOD, HERRON & EVANS, LLP, 2700 CAREW TOWER, 441 VINE STREET, CINCINNATI, OH, 45202, US

(73) Assignee: MEDEX, INC., Carlsbad, CA (US)

(21) Appl. No.: 10/905,047

(22) Filed: Dec. 13, 2004

(51) Int. Cl. A61M 31/00

(52) U.S. Cl. 604/65

(57) ABSTRACT

A bodily fluid space entry detection system includes electrical contacts in non-conducting relationship and associated with a needle cannula. Entry of the distal, sharp tip of the cannula into a bodily fluid space such as a blood vessel or epidural space allows bodily fluid such as blood or spinal fluid to make a conduction path between the contacts which, in turn, causes an alert source to be energized to indicate entry into the bodily fluid space. The alert source could be an LED and/or a buzzer.

### FIELD OF THE INVENTION

The present invention relates to insertion of cannula, such as needles or the like, into a bodily fluid space, and more particularly, to detection that the tip of the cannula has entered the bodily fluid space.

### DESCRIPTION OF PRIOR ART

In many situations, it is necessary that a needle or other cannula be inserted into a patient's body space which carries bodily fluid such as blood vessel carrying blood or an epidural space carrying spinal fluid. For example, when introducing a catheter into a patient's blood vessel for IV infusions and the like, a catheter with the sharp tip of a small gauge needle cannula extending therefrom is used to pierce the skin and the blood vessel so as to carry the end of the catheter into the vein. Once in place, the needle is withdrawn, leaving the catheter in place for administration or withdrawal of fluids, such as by connection with the catheter hub. As the needle enters into the blood vessel, blood will be forced back through or along a surface of the cannula into a chamber at the proximal end of the needle hub and/or catheter hub. This so-called flashback may be seen by the medical practitioner to know that the cannula has entered the vein. In some circumstances, the blood will not flash back quickly enough to be seen before the cannula tip has gone beyond the blood vessel, such as by coming out the other side of the vessel. This is particularly a concern with small gauge or long cannulas, or where there is a solid cannula with a grind or groove, or perhaps where a blunting device consumes part of the interior space of an otherwise hollow cannula.

One proposal has been to provide an optical lens system which allows the user to “see” blood at the proximal end of the cannula when the blood appears at the distal, sharp tip of the cannula. Such an optical lens system may be complex to manufacture, may not work well under certain conditions, or may require use of other equipment such as a separate light source. Others have proposed complex pressure sensing or acoustic sensing systems to more promptly detect entry into the bodily fluid space. These various proposals may not provide the desired results or may present other drawbacks.

### SUMMARY OF THE INVENTION

The present invention provides an improved bodily fluid space entry detection system and method in which entry of the cannula tip into the bodily fluid space is detected promptly and reliably and with the desired results. To this end, and in accordance with the principals of the present invention, entry of the cannula into a bodily fluid space is detected by energizing a visual and/or audible alert in response to the bodily fluid in the bodily fluid space establishing electrical conduction adjacent the cannula tip and/or along the cannula. In one aspect of the invention, a pair of electrical contacts are provided adjacent the cannula tip and/or along the cannula in non-conducting relationship such that upon entry into the bodily fluid space (such as a blood vessel or epidural space by way of example and not limitation), the bodily fluid normally present in the bodily fluid space (such as blood in the blood vessel or spinal fluid in the epidural space) establishes electrical conduction between the contacts. Detection of such conduction, such as by an alert circuit, causes an alert source to be energized to indicate to the medical practitioner that the cannula tip is in the bodily fluid space. The alert source may, for example, be an LED to provide a visual alert, and/or may be a buzzer to provide an audible alert.

The electrical contacts may be coupled to the alert circuit by first and second elongated conductors. One or both of the conductors may be insulated wires with respective portions, such as their respective distal ends, exposed to define the electrical contacts. Alternatively, one of the conductors may include the cannula itself, with the other conductor being an insulated wire or an insulated blunting member extending along the cannula with an exposed portion, such as the distal tip end. The cannula thus defines one of the electrical contacts with the exposed portion or tip of the insulated wire or blunting member defining the other electrical contact. The wire(s) may extend along the outer surface of the cannula, such as in a groove(s) in the outer surface thereof, or the insulated wire or insulated blunting member may extend through the cannula.

Advantageously, the pair of electrical contacts are adjacent the cannula tip. Where two wires are used, their exposed ends may be at or near the cannula tip. Where the cannula is one of the contacts, the area thereof adjacent the contact defined by the exposed portion of the wire or blunting member becomes the other contact. In that situation, where the wire extends to the cannula tip, the tip may be seen as the other contact.

In accordance with another aspect of the present invention, the alert circuit and alert source (such as the LED and/or buzzer) may be contained within the needle hub supporting the cannula, or may be contained in a housing attached to the needle hub, so as to be adjacent the proximal end of the cannula. Where a housing is used, the housing may be removably attached to the needle hub, so as to selectively connect to conductive elements communicating with the electrical contacts. A replaceable battery may also be included in the needle hub or housing.

In some circumstances, it may be desired to have multiple levels of alert. By way of example, while the cannula tip is being pushed into a blood vessel, for example, blood will continue to flow up into the needle cannula. However, if the needle tip progresses beyond the blood vessel, flow of blood into the cannula will be interrupted. In accordance with a yet further aspect of the present invention, multiple levels of alert are provided to indicate blood continuing to flow into the cannula, to thus imply that the needle tip is still within the blood vessel. To this end, and in accordance with this yet further aspect of the present invention, three or more electrical contacts are provided along a length of the cannula. As blood first enters into the cannula tip, a first conduction path is formed between the two contacts closest to the tip. That first conduction path is detected by the alert circuit and used to energize a first level of alert such as one of a plurality of lights, a particular intensity of light, or particular frequency or loudness of audible alert. As blood continues to enter up through the cannula, a second conduction path will form between two adjacent contacts further upstream from the cannula tip, which second conduction path can be detected by the alert circuit to energize a modified level of alert such as by energizing additional lights, changing the intensity of the light, or changing the frequency or loudness of the audible alert thus advising the user that blood is continuing to flow into the cannula. Should the needle tip pass completely through the blood vessel, flow of blood into the cannula will be interrupted before there would be additional changes in the alert signal, thus indicating that blood flow has discontinued.

The three or more electrical contacts may be provided the cannula and respective portions of a wire, by the cannula and two separate wires, or by multiple wires.

By virtue of the foregoing, there is thus provided an improved bodily fluid space entry detection system of method in which entry of the cannula tip into the bodily fluid space is detected promptly and reliably and with the desired results. These and other objects and advantages of the present invention shall be made apparent from the accompanying drawings and the description thereof.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is perspective view of a bodily fluid space entry detection system in a catheter arrangement entering a blood vessel in accordance with the principles of the present invention;

FIG. 2 is a partially cut-away, schematic view, not to scale, of a first embodiment of a bodily fluid space entry detection system, and which may be used in the catheter arrangement of FIG. 1;

FIG. 3 is a partially cut-away, schematic view, not to scale, of a second embodiment of a bodily fluid space entry detection system, and which may be used in the catheter arrangement of FIG. 1;

FIG. 4 is a schematic of an exemplary alert circuit and alert source of the bodily fluid space entry detection systems of FIGS. 1 through 3;

FIG. 5 is a perspective, partial view of an alternative conductor arrangement for a bodily fluid space entry detection system of the present invention;

FIG. 6 is a perspective, partial view of another catheter similar to FIG. 1 with a removable alert circuit housing and conductive elements therefor;

FIG. 7 is a perspective, partial view of a yet further alternative conductor arrangement for a bodily fluid space entry detection system of the present invention;

FIG. 8 is a perspective, partially cut away view, not to scale, of a portion of a third embodiment of a bodily fluid space entry detection system, and which may be used in the catheter arrangement of FIG. 1;

FIG. 9 is schematic, not to scale view of one embodiment of a bodily fluid space entry detection system for multiple level alerts; and

FIG. 10 is schematic, not to scale view of a second embodiment of a bodily fluid space entry detection system for multiple level alerts.

### DETAILED DESCRIPTION OF THE DRAWINGS

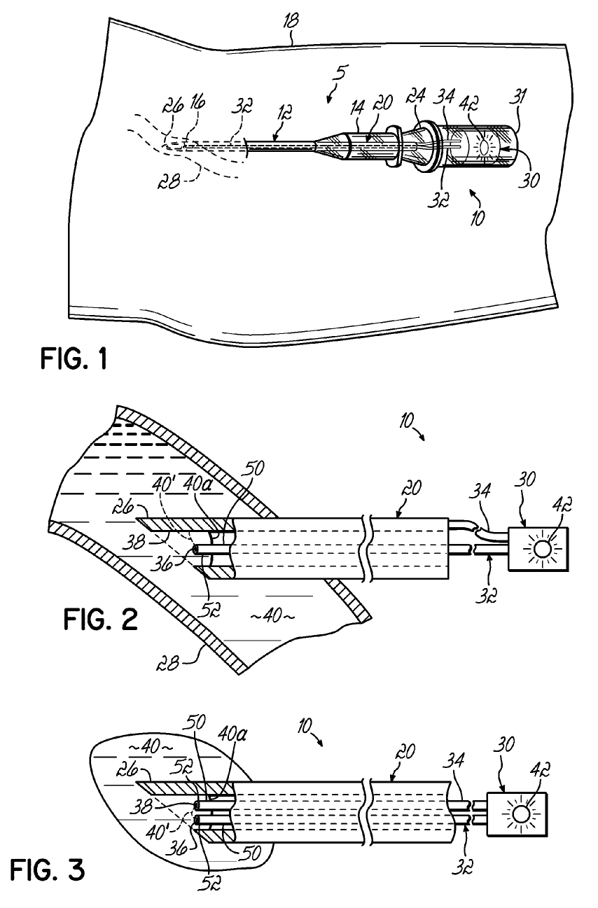

With reference to FIG. 1, there is shown a catheter introducer 5 assembled as a bodily fluid space entry detection system 10, in accordance with the principles of the present invention. System 10 will be described in connection with use for blood vessel entry detection, but it will be readily appreciated that the concepts described may be applied for entry detection of other bodily fluid spaces, one example of which is an epidural space. Catheter introducer 5 includes a catheter tube 12 extending from a proximal catheter hub 14 to a distal end 16 (shown in hidden line within a patient's arm 18). Introducer 5 also includes a needle cannula 20 which extends from a needle hub 24 (removably fitted to catheter hub 14) through catheter tube 12 to a distal, sharp tip end 26 (also shown in hidden line within arm 18 in FIG. 1). Tip 26 normally projects beyond catheter end 16 to pierce into the patient's skin 18 for entry into a bodily fluid space such as a blood vessel 28. Entry of sharp tip 26 into the blood vessel 28 carries with it catheter tip end 16 into vessel 28. The catheter hub 14 may be advanced to position catheter end 16 as desired within vessel 28, whereafter needle hub 24 may be separated from catheter hub 14 so as to withdraw needle cannula 20 from catheter tube 12 leaving the catheter tube 12 in place in the patient's blood vessel.

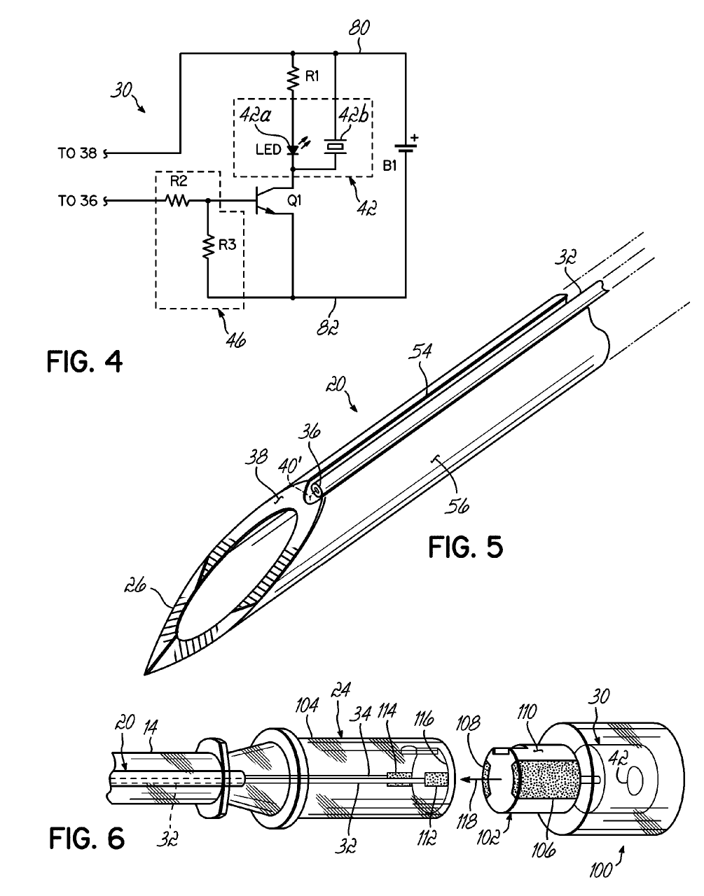

In order to determine entry into the blood vessel 28, it is advantageous to obtain a prompt indication that the needle tip 26 is in the blood vessel 28 and before the needle is pushed so far into the patient's arm 18 that it actually progresses beyond or through the patient's blood vessel 28. To this end, catheter introducer 5 is assembled as a bodily fluid space entry detection system 10 by inclusion of alert circuit 30, which may be provided within a translucent proximal housing portion 31 of needle hub 24, and is coupled via electrical insulated wires or conductors 32, 34 to communicate with electrical contacts 36, 38 (FIGS. 2 and 3 in which the catheter tube 12, hub 16, and needle hub 24 have been deleted for ease of viewing) which are associated with cannula 20. Contacts 36, 38 are shown adjacent the distal tip end 26 of needle cannula 20, but may alternatively be spaced away or upstream therefrom. Electrical contacts 36 and 38 are normally in non-conducting relationship (such as by being spaced apart) but with tip end 26 of needle cannula 20 in the blood vessel 28, blood 40 therein (FIGS. 2 and 3) will flow against and/or into tip 26 and appear between those contacts as at 40a creating an electrical conduction path represented schematically at 40′. Electrical conduction is detected by alert circuit 30 which causes an alert source 42 to be energized to notify the user (not shown) that tip end 26 is in the vessel 28. Alert source may advantageously present a visual alert, such as with an LED 42a (FIG. 4) (which may advantageously be a red LED as to simulate the appearance of blood in a flashback chamber) or may present an audible alert such as with a piezoelectric buzzer element 42b (FIG. 4), or both.

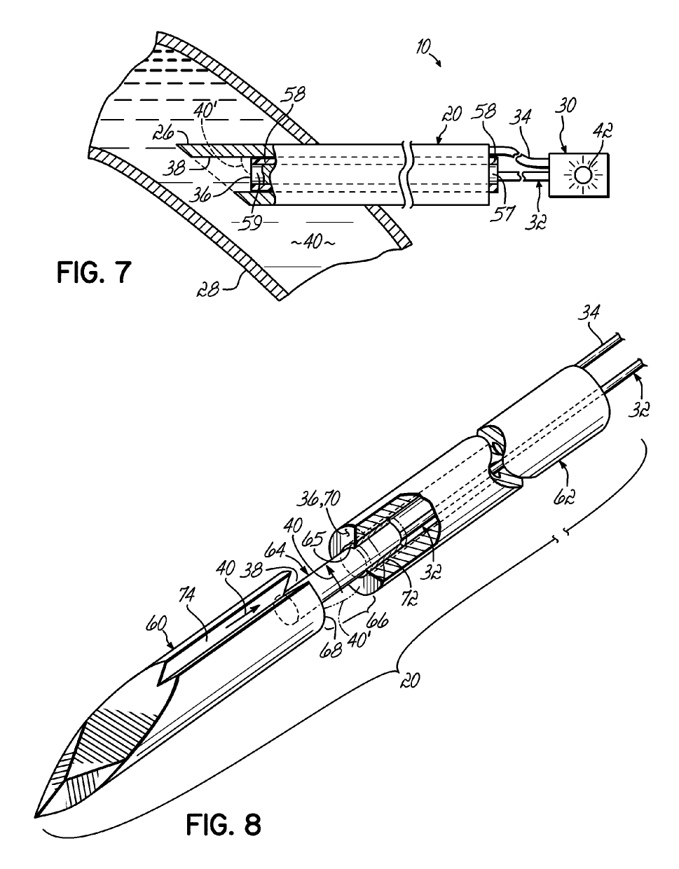

With further reference to FIG. 2, conductor 32 is an insulated wire extending along needle cannula 20 with the insulation 50 stopping at the distal end 52 of the wire. The exposed distal end 52 thus defines electrical contact 36. Alternatively, it will be appreciated that a portion of insulation 50 along wire 32 upstream of end 52 may be removed to expose a portion of the wire and define electrical contact 36 thereat. A typical needle cannula 20 is made of metal and thus is conductive. Accordingly, all or part of the other conductor 34 may be defined by the cannula 20 itself such that the cannula 20 defines, particularly near the electrical contact 36, the other electrical contact 38. In this arrangement, conductor 32 is insulated from but extends along cannula 20. Conductor 32 may extend through the interior of cannula 20 as seen in FIG. 2, or along a groove 54 formed in an outer surface 56 of cannula 20 as seen in FIG. 5, such that the electrical contact 36 is in non-conducting but closely spaced relationship to the other contact 38 defined by a portion of cannula 20 adjacent to contact 36, such as tip 26 of cannula 20. As seen in FIG. 7, conductor 32 may include, instead of an insulated wire, a conductive blunting member 57 extending through cannula 20, and which may have an insulating coating 58 such as Teflon® thereon, leaving the surface of distal tip 59 (or some other portion as desired) exposed to define contact 36. As will be readily apparent by those skilled in the art, blunt 57 is moveable within cannula 20 from a first position shown in FIG. 7 to a blunting position with distal tip 59 protruding from cannula tip 26 to thereby blunt the sharp tip 26. Blunting member 57 may be hollow or solid.

Alternatively, and as seen in FIG. 3, conductors 32 and 34 may both include insulated wires which extend along cannula 20, and advantageously through cannula 20 (FIG. 3) or along an outer surface 56 thereof such as in one or more grooves 54 (FIG. 5), toward the distal tip end 26 of cannula 20. The insulation 50 of conductor 34 also stops at distal end 52 thereon, or is removed from a portion thereof, to expose same and define electrical contact 38. Contacts 36 and 38 are shown to be adjacent tip 26 but one or both could be upstream thereof. They are in non-conducting relationship until blood 40 creates a conductive path 40′ therebetween. The wires of conductors 32 and 34 may extend alongside each other, may be a two-conductor cable, or may be twisted together along their length. Conductors 32, 34 may include magnet wire.

Cannula 20 is shown as being hollow but may also be solid, particularly where conventional blood flashback is not expected or required. However, where cannula 20 is hollow, blood 40 may still flow therethrough, and alongside the wire of conductor 32 (and 34 if it is present in the form of a wire extending through cannula 20) into the needle hub 24 and housing 31 thereof adjacent alert circuit 30 to thus provide conventional flashback as well.

With reference to FIG. 8, in which the catheter tube 12 and the hubs 14 and 24 are not shown for ease of viewing, needle cannula 20 may be defined by a solid, metal needle tip portion 60 and a metal cylinder 62 which are attached together via a post 64. Post 64 may be an integral part of tip portion 60 and received into a recess or opening 65 of cylinder 62 (or vice versa) so as to define a space or gap 66 between proximal end wall 68 of tip portion 60 and distal end wall 70 of cylinder 62. If post 64 is conductive, insulation 72 is provided to insulate post 64 from cylinder 62.

Wire 32 extends through cylinder 62 to and is coupled to tip portion 60 (such as via post 64) to define contact 38 at wall 68 and/or post 64. Cylinder 62 is part of conductor 34 such that distal end wall 70 defines the other contact 36. A groove 74 in tip portion 60 allows blood represented by arrow 40 to pass into gap 66 to create electrical conduction path 40′ between contacts 36 and 38 which are otherwise in non-conducting relationship.

With reference to FIG. 4, one exemplary alert circuit 30 is shown in schematic form, it being readily apparent to one of ordinary skill in the art that other electric circuitry may be utilized and organized to provide an alert circuit response to a conduction path 40′ appearing between contacts 36 and 38. Exemplary alert circuit 30 includes NPN transistor Q1, the collector of which is coupled through alert source 42 to the positive power supply rail 80 such as the positive terminal of battery B1, either directly or though 2.7 Kohm current-limiting resistor R1. The emitter of transistor Q1 is coupled to the negative supply rail 82 such as the negative terminal of battery B1. The base of terminal Q1 may be coupled directly to electrical contact 36 such as via conductor 32, or indirectly via a divider circuit 46 comprised of 1 Kohm series resistor R2 and 1 Mohm pull-down resistor R3. The other electrical contact 38 is coupled to the positive supply rail 80 such as via conductor 34. If battery B1 is a 3.0-volt battery, resistors R1, R2 and R3 are provided as described above. Alternatively, where battery B1 is of lower voltage, such as 1.5 volts, resistors R1 and R2 may be replaced with short circuits and resistor R3 may be eliminated.

In use, the sharp tip 26 is caused to pierce the patient's skin 18 and to be directed towards and advantageously into a bodily fluid space such as blood vessel 28. Before the needle tip 26 enters the blood vessel, electrical contacts 36 and 38 are in non-conducting relationship such that alert source 42 is not caused to be energized. However, upon entry into the blood vessel 28, blood 40 will appear across electrical contacts 36 and 38 to thus provide a conduction path 40′ (which may even be an electric short or of a low impedance). As a consequence, transistor Q1 will be activated thereby energizing alert source 42 to indicate to the user (not shown) that detection has been made of blood vessel entry. The user may discontinue insertion of the needle 20, may, if necessary, finish placing catheter tube 12, and may thereafter disconnect needle hub 24 from catheter hub 14 thereby withdrawing needle cannula 20 and exposing catheter hub 14 for subsequent use. Alternatively, the user may wait for a brief period to monitor for actual blood flashback into needle hub 24 before removal of the needle cannula 20.

In accordance with a further aspect of the present invention, alert circuit 30 and its associated alert source 42, and optionally battery B1, may be contained within a housing 100 which may be removably attached to needle hub 24 as shown in FIG. 6. Housing 100 includes a cylindrical stem portion 102 which is sized to snugly be received within cylinder 104 of needle hub 24 to thus make good physical contact therewith. Additionally, a pair of opposed conductive elements 106, 108 may be provided on or at the outer surface 110 of cylindrical portion 102 to mate with complimentary, opposed conductive elements 112, 114 on or at the inner surface 116 of needle hub cylinder 104. Elements 112, 114 are electrically coupled to conductors 32, 34 to thus complete the electrical connection to alert circuit 30, when housing 100 is inserted along the direction of arrow 118 into needle hub 24. The housing may be removed as necessary, such as for flushing or other purposes, and may even be reused with another catheter introducer device 5 or the like.

In accordance with a yet further aspect of the present invention, in some circumstances, it may be desired to have multiple levels of alert. By way of example, while the cannula tip is being pushed into the blood vessel, for example, blood will continue to flow up into the needle cannula. However, if the needle tip progresses beyond the blood vessel, flow of blood into the cannula will be interrupted. In accordance with a yet further aspect of the present invention, multiple levels of alert are provided to indicate blood continuing to flow into the cannula, and thus that the needle tip is still within the blood vessel. To this end, further electrical contacts beyond the pair of electrical contacts 36, 38 may be provided for detection of blood flow further up into the cannula.

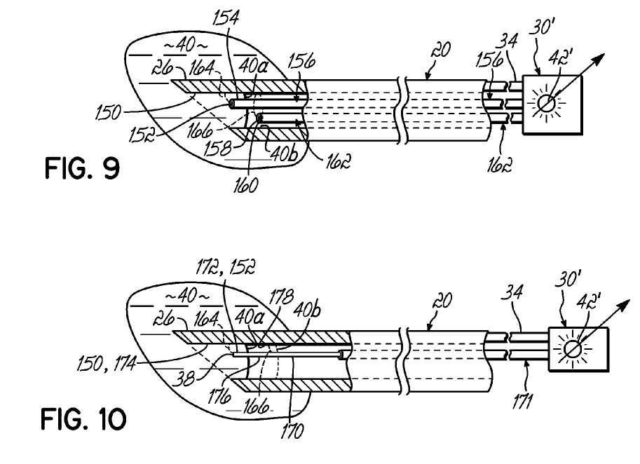

By way of example, and with reference to FIG. 9, a pair of electrical contacts 150, 152 are defined by cannula 20 and the exposed distal end 154 of insulated wire 156 extending through cannula 20, respectively. A third electrical contact 158 is defined by the exposed distal end 160 of another insulated wire 162 running alongside wire 156. The distal end 160 is upstream or further from tip 26 of cannula 20 than is distal end 154 of wire 156. Contacts 152 and 158 could, alternatively, be exposed portions along wires 156 and 162, respectively, spaced from their respective distal ends 154, 160. As blood 40 first enters into the cannula tip 26 as at 40a, a first conduction path 164 is formed between the electrical contact 150 of cannula 20 and electrical contact 152 closest to tip 26. Conduction path 164 is detected by alert circuit 30′ (which may be similar to or include multiple versions of alert circuit 30) and used to energize a variable alert signal 42′ which may have a first level of alert such as by illuminating one of a plurality of lights, illuminating a single light at a particular intensity, and/or emitting a particular frequency or loudness of audible alert. As blood 40 continues to enter up through the cannula 20 as at 40b, a second conduction path 166 is formed between either cannula 20 and upstream or third contact 158 or between contacts 152 and 158. Conduction path 166 is detected by the alert circuit 30′ to energize alert signal 42′ in a modified manner such as to cause the alert signal 42′ to provide a second level of alert such as by illuminating additional lights, changing the intensity of the light, and/or changing the frequency or loudness of the audible alert, thus advising the user that blood 40 is continuing to flow into the cannula 20. Further levels of alert could be provided if desired such as with further upstream electrical contacts (not shown). Should the needle tip 26 pass completely through the blood vessel 28, flow of blood 40 into the cannula 20 will be interrupted and there will be no additional changes in the alert signal 42′, thus indicating that blood flow has discontinued.

Another example of multiple level alert is shown in FIG. 10, wherein an elongated, uninsulated length 170 of wire 171 extends through, but in non-conducting relationship with, the cannula 20. As blood 40 first enters into the cannula tip 26 (as at 40a), first conduction path 164 is formed between a distal portion 172 of wire 171 and the adjacent portion 174 of the cannula 20. In that sense, distal portion 172 and cannula portion 174 define the pair of electrical contacts 150, 152 with conduction path 164 defining a first impedance level of those contacts. That impedance level is detected by the alert circuit 30′ and used to energize a variable alert signal 42′ with a first level of alert as above-described. As blood 40 continues to enter up through the cannula 20 (as at 40b), the conduction path 164 expands as at 166 to include an upstream portion 176 of the wire 171 and more of the cannula 20 as at 178, as if those further aspects were further electrical contacts included in the conduction path 164, thereby changing the effective impedance therebetween. That change in impedance is detected by the alert circuit 30′ to energize alert signal 42′ in a modified manner such as to cause the alert signal 42′ to provide the second level of alert as also described above. Further levels of alert could be provided if desired. Should the needle tip 26 pass completely through the blood vessel 28, flow of blood 40 into the cannula 20 will be interrupted and there will be no additional changes in the alert signal 42′, thus indicating that blood flow has discontinued.

By virtue of the foregoing, there is thus provided an improved bodily fluid space entry detection system and method in which entry of the cannula tip into the bodily fluid space is detected promptly and reliably and with the desired results.

While the present invention has been illustrated by the description of embodiments thereof, and while the embodiments have been described in considerable detail, it is not intended to restrict or in any way limit the scope of the appended claims to such detail. Additional advantages and modifications will readily appear to those skilled in the art. For example, the embodiments are described here in the context of a blood vessel carrying blood, the invention may be applied to other bodily fluid spaces such as an epidural space carrying spinal fluid or other bodily fluid spaces to be accessed by a cannula or catheter. Moreover, while shown as part of a catheter introducer device 5, the bodily fluid space entry detection system may be deployed for a needle cannula 20 without a catheter, such as in the case of hypodermic or other needles. Further, while contacts 36 and 38 are generally shown as being adjacent the needle tip 26, it will be appreciated that “adjacent” in this context may also include “at” or “in” the needle tip 26. And although in some embodiments the cannula provides one of the contacts, and an insulated wire or blunting member provides the other, various other arrangements are possible. For example, a conductive, non-insulated blunt could be used. In that arrangement, one of the contacts and conductor therefor would be provided by an insulated wire extending through or along an outer surface of the blunting member, with the blunting member or the combination of the blunting member and the cannula defining the other of the contacts and part of the associated conductor. The invention in its broader aspects is, therefore, not limited to the specific details, representative apparatus and method, and illustrative examples shown and described. Accordingly, departures may be made from such details without departing from the spirit or scope of the general inventive concept.

1. A bodily fluid space entry detection system comprising:
a cannula having a proximal end and a distal end, the distal end defining a sharp tip adapted to enter a bodily fluid space;
a pair of electrical contacts in non-conductive relationship associated with the cannula;
an electrically energizable alert source; and
an alert circuit in electrical communication with the pair of electrical contacts and adapted to cause the alert source to be electrically energized in response to bodily fluid establishing electrical conduction between the pair of electrical contacts whereby to provide an indication that the cannula tip is in a bodily fluid space.

2. The bodily fluid space entry detection system of claim 1 further comprising a first electrical conductor extending along a length of the cannula with a portion of the first electrical conductor exposed adjacent the cannula and defining one of the pair of electrical contacts.

3. The bodily fluid space entry detection system of claim 2, the exposed portion of the first electrical conductor being adjacent the cannula tip.

4. The bodily fluid space entry detection system of claim 2, the cannula including a groove on an exterior surface thereof, the first electrical conductor extending in the groove.

5. The bodily fluid space entry detection system of claim 2, the first electrical conductor extending within the cannula.

6. The bodily fluid space entry detection system of claim 5, the first electrical conductor including a conductive blunting member moveable within the cannula.

7. The bodily fluid space entry detection system of claim 6, further comprising an insulating coating on the conductive blunting member.

8. The bodily fluid space entry detection system of claim 2 further comprising a second electrical conductor extending along a length of the cannula with a portion of the second electrical conductor exposed adjacent the cannula and defining another of the pair of electrical contacts.

9. The bodily fluid space entry detection system of claim 8, the exposed portions of the first and second electrical conductors each being adjacent the cannula tip.

10. The bodily fluid space entry detection system of claim 8, one of the pair of electrical contacts being spaced further from the cannula tip then the other of the pair of electrical contacts.

11. The bodily fluid space entry detection system of claim 8, the conductors extending within the cannula.

12. The bodily fluid space entry detection system of claim 8, the electrical conductors being insulated from each other except at their respective exposed portions.

13. The bodily fluid space entry detection system of claim 2 wherein the cannula is conductive and defines another of the pair of electrical contacts.

14. The bodily fluid space entry detection system of claim 13, the first electrical conductor being insulated from the cannula except at a portion thereof.

15. The bodily fluid space entry detection system of claim 14, the portion being a distal end of the first electrical contact.

16. The bodily fluid space entry detection system of claim 14, the cannula including a groove on an exterior surface thereof, the first electrical conductor extending in the groove.

17. The bodily fluid space entry detection system of claim 13 further comprising a conductive blunting member moveable within the cannula.

18. The bodily fluid space entry detection system of claim 1, the alert source including an LED.

19. The bodily fluid space entry detection system of claim 18, the LED being red.

20. The bodily fluid space entry detection system of claim 18, the alert source further including a buzzer.

21. The bodily fluid space entry detection system of claim 1, the alert source including a buzzer.

22. The bodily fluid space entry detection system of claim 1 further comprising a battery.

23. The bodily fluid space entry detection system of claim 1, the alert source being positioned near the proximal end of the cannula.

24. The bodily fluid space entry detection system of claim 1, the alert circuit being positioned near the proximal end of the cannula.

25. The bodily fluid space entry detection system of claim 1 further comprising a housing containing the alert circuit and the alert source.

26. The bodily fluid space entry detection system of claim 25, the housing further containing a battery.

27. The bodily fluid space entry detection system of claim 26, the housing being removably connected to conductive elements electrically communicating with the electrical contacts.

28. The bodily fluid space entry detection system of claim 25, the housing being removably connected to conductive elements electrically communicating with the electrical contacts.

29. The bodily fluid space entry detection system of claim 1, further comprising a conductive blunting member moveable within the cannula.

30. The bodily fluid space entry detection system of claim 26, further comprising an insulating coating on the conductive blunting member.

31. The bodily fluid space entry detection system of claim 1 further comprising a third electrical contact associated with the cannula and being in non-conducting relationship with at least a first of the pair of electrical contacts, the alert circuit further being in electrical communication with the third electrical contact and adapted to cause the alert source to be electrically energized in response to bodily fluid establishing electrical conduction between the third and the at least first of the pair of electrical contacts.

32. The bodily fluid space entry detection system of claim 31 further comprising a conductive wire defining the third electrical contact and a second of the pair of the electrical contacts.

33. The bodily fluid space entry detection system of claim 1 further comprising at least a third electrical contact associated with the cannula and being in non-conducting relationship with both of the pair of electrical contacts, the alert circuit further being in electrical communication with the third electrical contact and adapted to cause the alert source to be electrically energized in response to bodily fluid establishing electrical conduction between the third electrical circuit and at least one of the pair of electrical contacts.

34. A catheter introducer device comprising:
a catheter hub and a catheter tube extending therefrom;
a cannula having a proximal end and a distal end, the distal end defining a sharp tip adapted to enter a bodily fluid space, the cannula extending through the catheter tube with the cannula tip projecting out therefrom;
a pair of electrical contacts in non-conductive relationship associated with cannula;
an electrically energizable alert source; and
an alert circuit in electrical communication with the pair of electrical contacts and adapted to cause the alert source to be electrically energized in response to body fluid establishing electrical conduction between the pair of electrical contacts whereby to provide an indication that the cannula tip is in a bodily fluid space.

35. The catheter introducer device of claim 34 further comprising a first electrical conductor extending along a length of the cannula with a portion of the first electrical conductor exposed adjacent the cannula and defining one of the pair of electrical contacts.

36. The catheter introducer device of claim 35, the exposed portion of the first electrical conductor being adjacent the cannula tip.

37. The catheter introducer device of claim 35, the cannula including a groove on an exterior surface thereof, the first electrical conductor extending in the groove.

38. The catheter introducer device of claim 35, the first electrical conductor extending within the cannula.

39. The catheter introducer device of claim 38, the first electrical conductor including a conductive blunting member moveable within the cannula.

40. The catheter introducer device of claim 39, further comprising an insulating coating on the conductive blunting member.

41. The catheter introducer device of claim 35 further comprising a second electrical conductor extending along a length of the cannula with a portion of the second electrical conductor exposed adjacent the cannula and defining another of the pair of electrical contacts.

42. The catheter introducer device of claim 41, the exposed portions of the first and second electrical conductors each being adjacent the cannula tip.

43. The catheter introducer device of claim 41, one of the pair of electrical contacts being spaced further from the cannula tip then the other of the pair of electrical contacts.

44. The catheter introducer device of claim 41, the conductors extending within the cannula.

45. The catheter introducer device of claim 41, the electrical conductors being insulated from each other except at their respective exposed portions.

46. The catheter introducer device of claim 35 wherein the cannula is conductive and defines another of the pair of electrical contacts.

47. The catheter introducer device of claim 46, the first electrical conductor being insulated from the cannula except at a portion thereof.

48. The catheter introducer device of claim 47, the portion being a distal end of the first electrical conductor.

49. The catheter introducer device of claim 47, the cannula including a groove on an exterior surface thereof, the first electrical conductor extending in the groove.

50. The catheter introducer device of claim 46, further comprising a conductive blunting member moveable within the cannula.

51. The catheter introducer device of claim 34, the alert source including an LED.

52. The catheter introducer device of claim 51, the LED being red.

53. The catheter introducer device of claim 51, the alert source further including a buzzer.

54. The catheter introducer device of claim 34, the alert source including a buzzer.

55. The catheter introducer device of claim 34 further comprising a battery.

56. The catheter introducer device of claim 34, the alert source being positioned near the catheter hub.

57. The catheter introducer device of claim 34, the alert circuit being positioned near the catheter hub.

58. The catheter introducer device of claim 34 further comprising a housing containing the alert circuit and the alert source.

59. The catheter introducer device of claim 58, the housing further including a battery.

60. The catheter introducer device of claim 59, the housing being removably mounted to the catheter hub.

61. The catheter introducer device of claim 59, the housing being removably connected to conductive elements electrically communicating with the electrical contacts.

62. The catheter introducer device of claim 61, one of the conductive elements communicating with one of the electrical contacts via the cannula.

63. The catheter introducer device of claim 61, one of the conductive elements communicating with one of the electrical contacts via a blunting member.

64. The catheter introducer device of claim 58, the housing being removably mounted to the catheter hub.

65. The catheter introducer device of claim 58, the housing being removably connected to conductive elements electrically communicating with the electrical contacts.

66. The catheter introducer device of claim 65, one of the conductive elements communicating with one of the electrical contacts via the cannula.

67. The catheter introducer device of claim 65, one of the conductive elements communicating with one of the electrical contacts via a blunting member.

68. The catheter introducer device of claim 34 further comprising a third electrical contact associated with the cannula and being in non-conducting relationship with at least a first of the pair of electrical contacts, the alert circuit further being in electrical communication with the third electrical contact and adapted to cause the alert source to be electrically energized in response to bodily fluid establishing electrical conduction between the third and the at least first of the pair of electrical contacts.

69. The catheter introducer device of claim 68 further comprising a conductive wire defining the third electrical and a second of the pair of electrical contacts.

70. The catheter introducer device of claim 34 further comprising at least a third electrical contact associated with the cannula and in non-conducting relationship with both of the pair of electrical contacts, the alert circuit further being in electrical communication with the third electrical contact and adapted to cause the alert source to be electrically energized in response to bodily fluid establishing electrical conduction between the third electrical circuit and at least one of the pair of electrical contacts.

71. A method of detecting entry of a cannula into a bodily fluid space comprising inserting a tip of a cannula into the bodily fluid space and energizing an alert source in response to body fluid in the bodily fluid space establishing electrical conduction along the cannula.

72. The method of claim 71 wherein energizing an alert source includes energizing an LED.

73. The method of claim 71 wherein energizing an alert source includes energizing a buzzer.

74. The method of claim 71 wherein the cannula extends through a catheter to be introduced into the bodily fluid space, the method further comprising withdrawing the cannula from the catheter after the alert source is energized.

75. The method of claim 71 wherein the alert source is coupled to an alert circuit and a battery all contained within a housing removably held adjacent an end of the cannula opposite the tip, the method further comprising removing the housing.

76. The method of claim 71 wherein the alert source is energized in response to the bodily fluid establishing electrical contact adjacent the cannula tip.

77. The method of claim 76 wherein the bodily fluid space is a blood vessel, the method further comprising inserting the cannula tip into the blood vessel and energizing the alert source in response to blood establishing the electrical conduction.

78. The method of claim 71, wherein the bodily fluid space is a blood vessel, the method further comprising inserting the cannula tip into the blood vessel and energizing the alert source in response to blood establishing the electrical conduction.

79. The method of claim 71 further comprising energizing the alert source in a modified manner in response to modified electrical conduction being established along the cannula by further flow of bodily fluid along the cannula.
